(19) United States
(12) Patent Application Publication
(10) Pub. No.: US 2019/0345551 A1
(43) Pub. Date: Nov. 14, 2019

(54) METHOD FOR DETECTING MITOCHONDRIAL tRAN MODIFICATION

(71) Applicant: NATIONAL UNIVERSITY CORPORATION KUMAMOTO UNIVERSITY, Kumamoto (JP)

(72) Inventors: Kazuhito TOMIZAWA, Kumamoto (JP); Fanyan WEI, Kumamoto (JP)

(21) Appl. No.: 16/474,969

(22) PCT Filed: Dec. 27, 2017
(86) PCT No.: PCT/JP2017/047099

§ 371 Date: Jun. 28, 2019

(30) Foreign Application Priority Data
Dec. 28, 2016 (JP) 2016-255808

(51) Int. Cl. C12Q 1/6872; G01N 30/72; G01N 33/493

(52) U.S. Cl. CPC C12Q 1/6872; G01N 30/7266; G01N 2030/027; G01N 2560/00; G01N 33/493

(57) ABSTRACT

An exemplary method can be provided for detecting a modified nucleoside of mitochondrial transition RNA (mt-tRNA). Another exemplary method can be provided for detecting a modified nucleoside in mt-tRNA, which can use tandem mass analysis. For exemplary, it is possible to detect a modified nucleoside (for example, 5-taurinomethyl-2-thiouridine (τm5s2U), 5-taurinomethyluridine (τm5U), 2-methylthio-N6-isopentenyl adenosine (ms2i6A)) in a body fluid sample such as urine or a sample of cell culture supernatant.

### FIELD OF THE DISCLOSURE

The present disclosure relates to a method for detecting mitochondrial transfer RNA (mt-tRNA) modification. Specifically, the present disclosure relates to a method for detecting modified nucleosides in a sample using tandem mass spectrometry, a method for detecting modified nucleosides in mt-tRNA characterized by detecting modified nucleosides in a body fluid sample such as urine or the culture supernatant, for example, taurine-modified uridine (5-taurinomethyl-2-thiouridine (τm⁵ s²U) or 5-taurinomethyluridine (τm⁵U)) and 2-methylthio-N6-isopentenyl adenosine (ms² i⁶A), a method of diagnosing a mitochondrial disease using this method, and the like.

### BACKGROUND INFORMATION

There are 22 types of transfer RNA (tRNA) derived from mitochondrial DNA in mitochondria, and 13 types of proteins also derived from mitochondrial DNA are essential for translation. mt-tRNA is known to contain many chemical modifications, and so far, a total of 15 types of chemical modifications have been identified at 118 base positions among the bases constituting mt-tRNA. For example, of the mt-tRNAs, five tRNAs contain taurine modification at the 34th uridine. Among them, mt-tRNA^{Leu} and mt-tRNA^{Trp} are taurinomethylated (τm⁵U) and mt-tRNA^{Gln}, mt-tRNA^{Glu} and mt-tRNA^{Lys} are taurinomethylthiolated (τm⁵s²U) (FIG. 1). Further, in mt-tRNA^{Trp}, the 37th adenosine is also modified to be ms²i⁶A.

The importance of taurine modification in mitochondria has been suggested from findings in mitochondrial disease patients (Non-Patent Document 1). In addition, ms²i⁶A has also been suggested to be correlated with mitochondrial diseases. Mitochondrial diseases are genetic diseases that are mainly caused by point mutations in mitochondrial DNA and cause damage to the heart muscle and skeletal muscle with high energy demand. As an example, among mitochondrial DNA point mutations, the frequency of the A3243G point mutation generated in the DNA region encoding mt-tRNA^{Leu} and the A8344G point mutation generated in the DNA region encoding mt-tRNA^{Lys} is particularly high. Interestingly, in patients with the A3243G point mutation, the τm⁵ modification of mt-tRNA^{Leu} was abolished. In addition, even in patients with a mitochondrial disease having the A8344G point mutation, the τm⁵s² modification of mt-tRNA^{Lys} was abolished. From these facts, it is strongly suggested that the reduction of taurine modification is the cause of the onset of mitochondrial diseases.

That is, mitochondrial diseases can be diagnosed by analyzing the amount of modified nucleosides typically including taurine-modified uridine. However, in the prior art, for example, in order to analyze taurine-modified uridine, it is necessary to collect a large amount of muscle tissue from a patient, and this method cannot be applied to the diagnosis of a mitochondrial disease which often occurs in children.

### OBJECTS AND SUMMARY OF EXEMPLARY EMBODIMENTS

An object of the present disclosure is, for example, to provide a method for detecting modified nucleosides such as taurine-modified uridine and the like.

According to exemplary embodiments of the present disclosure, it is possible to detect τm⁵s²U or τm⁵U by decomposing RNA samples obtained from embryonic stem (ES) cells into nucleosides by an enzymatic treatment, and subjecting the resulting nucleosides to liquid chromatography followed by tandem mass spectrometry. In addition, when ES cells (Mto1-KO ES cells) in which taurine modification of mt-tRNA is inhibited were prepared and the similar analysis was performed using RNA samples obtained from the cells, no similar signal was observed. Therefore, it was shown that the signal detected by this detection method is specific to τm⁵s²U or τm⁵U. Furthermore, the present inventors have found that by using an RNA sample obtained from the culture supernatant of cells or urine, τm⁵s²U or τm⁵U can be detected, without through the step of decomposing the RNA sample.

The basis of the exemplary embodiments have been analyzed to complete the exemplary embodiments of the present disclosure.

That is, the present disclosure includes the following exemplary embodiments.

{1} A method for determining the amount of modified nucleosides contained in mitochondrial tRNA (mt-tRNA) in a test animal, comprising

(i) a determining the amount of modified nucleosides in a sample selected from the group consisting of a body fluid sample derived from the test animal and a culture supernatant of cells derived from the test animal using tandem mass spectrometry; and

(ii) coorellating the amount of modified nucleosides in the sample determined by the step (i) with the amount of said modified nucleosides in mt-tRNA in the test animal.

{2} The method according to {1}, wherein said tandem mass spectrometry is LC-ESI-MS/MS, which includes liquid chromatography (LC) as a pretreatment and in which the ionization source is an electrospray ionization source (ESI), and the mass spectrometry mode to be used is selective reaction monitoring.

{3} The method according to {1} or {2}, wherein said sample includes urine derived from a test animal.

{4} The method according to {3}, wherein said sample is a urine sample deproteinized by methanol.

{5} The method according to any one of {1} to {4}, wherein said sample is a urine sample from a human suspected of having a mitochondrial disease.

{6} The method according to any one of {1} to {5}, further comprising

(iii) correlating the amount of modified nucleosides in mt-tRNA in a test animal correlated according to step (ii) with the degree of a mitochondrial disease in the test animal.

{7} The method according to any one of {1} to {5}, wherein said modified nucleoside is taurine-modified uridine.

{8} The method according to {7}, wherein said taurine-modified uridine is 5-taurinomethyl 2-thiouridine (τm⁵s²U).

{9} The method according to {8}, wherein said step (1) comprises

(a) subjecting a sample suspected to contain τm⁵s²U to liquid chromatography to obtain a fraction enriched in τm⁵s²U,

(b) subjecting the fraction enriched in τm⁵s²U to an ionization source under conditions suitable to generate a τm⁵s²U precursor ion that can be detected by mass spectrometry, and

(c determining the amount of τm⁵s²U generated ions by tandem mass spectrometry (MS/MS),

wherein the tandem mass spectrometry (MS/MS) in the step (c) includes subjecting the precursor negative ions having a mass-to-charge ratio (m/z) of 396±0.5 to a collision reaction under the condition wherein the τm⁵s²U precursor negative ions generate τm⁵s²U generated negative ions having an m/z of 124±0.5, to determine the amount of the generated ions having an m/z of 124±0.5 generated by the collision reaction

and the amount of ions determined by the step (c) is correlated with the amount of τm⁵s²U in said sample.

{10} The method according to {9}, wherein said sample is a urine sample obtained by deproteinizing urine derived from a test animal with methanol.

{11} The method according to {7}, wherein said taurine-modified uridine is 5-taurinomethyluridine (τm5U).

{12} The method according to {11}, wherein said step (1) comprises

(i) subjecting a sample suspected to contain τm⁵U to liquid chromatography to obtain a fraction enriched in τm⁵U,

(ii) subjecting the fraction enriched in τm⁵U to an ionization source under conditions suitable to generate a τm⁵U precursor ion that can be detected by mass spectrometry, and

(iii) determining the amount of τm⁵U generated ions by tandem mass spectrometry (MS/MS),

wherein the tandem mass spectrometry (MS/MS) in the step (c) includes subjecting the precursor negative ions having a mass-to-charge ratio (m/z) of 380±0.5 to a collision reaction under the condition wherein the τm⁵U precursor negative ions generate τm⁵U generated negative ions having an m/z of 124±0.5, to determine the amount of the generated ions having an m/z of 124±0.5 generated by the collision reaction

and the amount of ions determined by the step (iii) is correlated with the amount of τm⁵U in said sample.

{13} The method according to {12}, wherein said sample is a urine sample obtained by deproteinizing urine derived from a test animal with methanol.

{14} The method according to any one of {1} to {5}, wherein said modified nucleoside is 2-methylthio-N6-isopentenyl adenosine (ms²i⁶A).

{15} The method according to {14}, wherein said step (i) comprises

(a) subjecting a sample suspected to contain ms²i⁶A to liquid chromatography to obtain a fraction enriched in ms²i⁶A,

(b) subjecting the fraction enriched in ms²i⁶A to an ionization source under conditions suitable to generate a ms²i⁶A precursor ion that can be detected by mass spectrometry, and

(c) determining the amount of ms²i⁶A generated ions by tandem mass spectrometry (MS/MS),

wherein the tandem mass spectrometry (MS/MS) in the step (c) includes subjecting the precursor positive ions having a mass-to-charge ratio (m/z) of 382±0.5 to a collision reaction under the condition wherein the ms²i⁶A precursor positive ions generate ms²i⁶A generated positive ions having an m/z of 182±0.5, to determine the amount of the generated ions having an m/z of 182±0.5 generated by the collision reaction

and the amount of ions determined by the step (c) is correlated with the amount of ms²i⁶A in said sample.

{16} The method according to {15}, wherein said sample is a urine sample obtained by deproteinizing urine derived from a test animal with methanol.

The present disclosure further includes the following further exemplary embodiments.

[1] A method for determining the amount of τm⁵s²U in a sample by tandem mass spectrometry, comprising

(a) subjecting a sample suspected to contain τm⁵s²U to liquid chromatography to obtain a fraction enriched in τm⁵s²U,

(b) subjecting the fraction enriched in τm⁵s²U to an ionization source under conditions suitable to generate a τm⁵s²U precursor ion that can be detected by mass spectrometry, and

(c) determining the amount of τm⁵s²U generated ions by tandem mass spectrometry,

wherein the tandem mass spectrometry in the step (c) includes subjecting the precursor negative ions having a mass-to-charge ratio (m/z) of 396±0.5 to a collision reaction under the condition wherein the τm⁵s²U precursor negative ions generate τm⁵s²U generated negative ions having an m/z of 124±0.5, to determine the amount of the generated ions having an m/z of 124±0.5 generated by the collision reaction

and the amount of ions determined by the step (c) is correlated with the amount of τm⁵s²U in said sample.

[2] A method for determining the amount of τm⁵U in a sample by tandem mass spectrometry, comprising

(a) subjecting a sample suspected to contain τm⁵U to LC to obtain a fraction enriched in τm⁵U,

(b) subjecting the fraction enriched in τm⁵U to an ionization source under conditions suitable to generate a τm⁵U precursor ion that can be detected by mass spectrometry, and

(c) determining the amount of τm⁵U generated ions by tandem mass spectrometry,

wherein the tandem mass spectrometry in the step (c) includes subjecting the precursor negative ions having a mass-to-charge ratio (m/z) of 380±0.5 to a collision reaction under the condition wherein the τm⁵U precursor negative ions generate τm⁵U generated negative ions having an m/z of 124±0.5, to determine the amount of the generated ions having an m/z of 124±0.5 generated by the collision reaction

and the amount of ions determined by the step (c) is correlated with the amount of τm⁵U in said sample.

[3] The method according to any one of [1] or [2], wherein said ionization source is an electrospray ionization source.

[4] The method according to any one of [1] to [3], wherein the sample includes a body fluid sample or cell culture supernatant.

[5] The method according to any one of [1] to [3], wherein the sample includes urine.

[6] The method according to any one of [1] to [3], wherein the sample is a deproteinized body fluid sample or cell culture supernatant.

[7] A method for determining the amount of taurine-modified uridine contained in mitochondria (mt)-tRNA in a test animal, comprising determining the amount of taurine-modified uridine in a sample selected from the group consisting of a culture supernatant of cells derived from the test animal and a body fluid sample derived from the test animal, wherein the determined amount of taurine-modified uridine in the sample is correlated with the amount of taurine-modified uridine in the test animal.

[8] The method according to [7], wherein the sample is a body fluid sample.

[9] The method according to [7], wherein the sample is a urine sample.

[10] The method according to any one of [7] to [9], wherein the taurine-modified uridine is τm⁵s²U.

[11] The method according to [10], wherein the determination of the amount of taurine-modified uridine is performed using the method according to [1].

[12] The method according to any one of [7] to [9], wherein the taurine-modified uridine is τm⁵U.

[13] The method according to [12], wherein the determination of the amount of taurine-modified uridine is performed using the method according to [2].

[14] The method according to [9], wherein the method for determining the amount of taurine-modified uridine in a sample comprises

(a) subjecting 1 to 100 μL of a deproteinized urine sample to liquid chromatography (LC), to obtain a fraction enriched in taurine-modified uridine,

(b) subjecting the fraction enriched in taurine-modified uridine to an ionization source under conditions suitable to generate a taurine-modified uridine ion that can be detected by mass spectrometry, and

(c) determining the amount of taurine-modified uridine ions by mass spectrometry, wherein the amount of ions determined by step (c) is correlated with the amount of taurine-modified uridine in the sample.

[15] The method according to any one of [9] to [14], wherein the sample is a urine sample deproteinized by methanol.

[16] A method of diagnosing a mitochondrial disease, comprising determining the amount of taurine-modified uridine contained in mt-tRNA in a test animal by the method according to any one of [7] to [14].

According to exemplary embodiments the method for detecting a modified nucleoside, for example, taurine-modified uridine or 2-methylthio-N6-isopentenyl-modified (ms²i⁶-modified) adenosine according to the present disclosure, it is possible to specifically detect modified nucleosides such as taurine-modified uridine and the like from a sample containing various substances including a body fluid sample such as urine and the cell culture supernatant. Furthermore, according to the exemplary embodiments of the method for detecting a modified nucleoside such as taurine-modified uridine and the like in a test animal provided in the present disclosure, it may be possible to non-invasively detect a modified nucleoside such as taurine-modified uridine and the like from a biological sample such as urine, without conducting painful muscle biopsy. Moreover, according to the exemplary embodiments of the detection method, it is possible to detect a modified nucleoside such as taurine-modified uridine and the like from a small amount of sample and all pretreatments are completed with just a few steps of centrifugation and concentration, thus, the method is very simple, the cost thereof is low, and anyone can get stable results. Furthermore, functional analysis of mitochondria and diagnosis of mitochondrial diseases may be possible by using the method for detecting a modified nucleoside such as taurine-modified uridine and the like provided in the present disclosure.

The aspects described above and further aspects, features and advantages of the present disclosure may also be found in the exemplary embodiments which are described in the following with reference to the appended drawings and claims.

### BRIEF DESCRIPTION OF THE DRAWINGS

Further exemplary embodiments of the present disclosure are detailed in the description of the Figures, where this description shall not limit the scope of the present disclosure. The Figures show that:

FIG. 1 is a diagram showing the sequence of a mitochondrial tRNA containing taurine modification. In mammals, five types of mt-tRNA have taurine modification. Mitochondrial tRNA^{Leu(UUR)} and tRNA^{Lys} contain taurinomethyluridine (τm⁵U) at the position 34 of the anticodon, and mitochondrial tRNA^{TrP}, tRNA^{Gln} and tRNA^{Glu} contain taurinenomethylthioluridine (τm⁵s²U) at the position 34 of the anticodon. Further, mt-tRNA^{Trp} contains ms²i⁶A at the position 37.

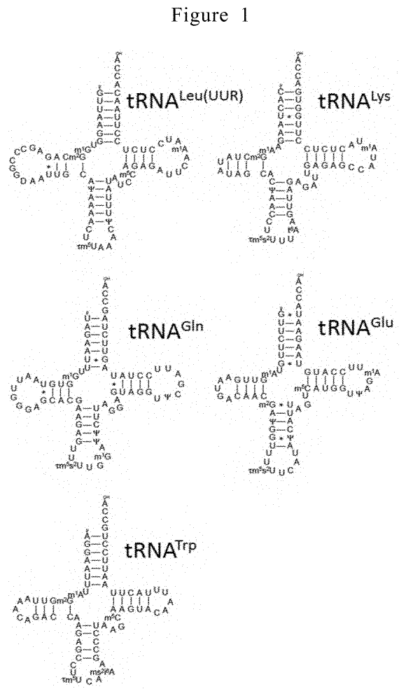

FIG. 2 is a diagram illustrating a conventional method for detecting taurine-modified uridine.

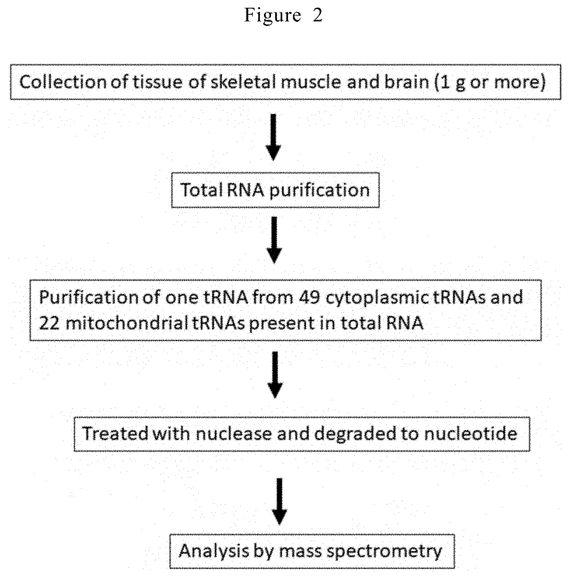

FIG. 3 is a diagram showing the flow of detection of taurine modification in a trace sample. Deproteinization is carried out by adding 500 μL of methanol to 100 μL of culture supernatant or human urine sample, then centrifuging at 15000 rpm for 10 minutes and recovering the supernatant. The collected supernatant is dried in a centrifugal evaporator. Further, the precipitate is dissolved in 100 μL of distilled water, and 2 μL thereof is analyzed by a tandem mass spectrometer (LCMS-8050 manufactured by Shimadzu Corporation).

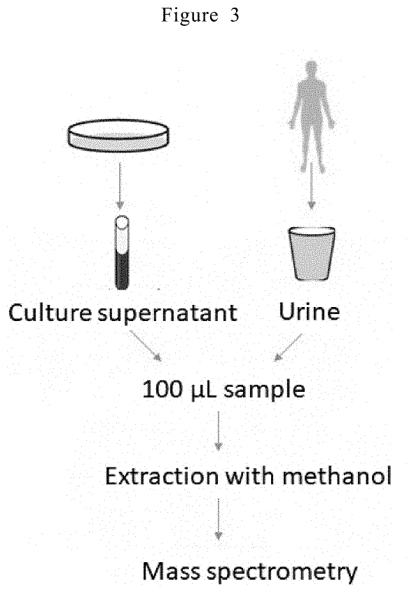

FIG. 4 is a diagram showing the validation of the taurine modification detection method by tandem mass spectrometry. In order to validate the detection method of τm⁵U and τm⁵s²U by mass spectrometry with selective reaction monitoring, total RNA was extracted from wild type ES cells and ES cells deficient in the taurine modification enzyme Mto1 (mitochondrial translation optimization 1) and analyzed by a tandem mass spectrometer. The peak of τm⁵s²U was detected 3 minutes after the start of analysis, and the peak of tm⁵U was detected 2 minutes after the start of analysis. Since respective peaks were not found in RNA derived from Mto1-KO cells containing no taurine modification, it was validated that the peaks observed at 3 and 2 minutes corresponded to τm⁵s²U and τm⁵U, respectively. Parameters for the selective reaction monitoring are as follows. τm⁵U: precursor ion m/z 380, product ion m/z 124; τm⁵s²U: precursor ion m/z 396, product ion m/z 124.

FIG. 5 is a diagram showing the detection of τm⁵U and τm⁵s²U in the culture supernatant. HeLa cells (1.5×10⁵ cells), which are human-derived culture cells, were seeded in a 3.5 cm-diameter culture dish and cultured in 2 mL of DMEM medium overnight. Next, 100 μL of the cultured medium was subjected to methanol extraction, and 2 μL thereof was analyzed by a mass spectrometer. Peaks corresponding to τm⁵s²U and τm⁵U were detected.

FIG. 6 is a diagram showing the detection of τm⁵U and τm⁵s²U in human urine.

FIG. 7 is a diagram showing the results of comparison of the amount of taurine-modified uridine in urine samples of patients with a mitochondrial disease (MERRF) and healthy individuals. Arrows indicate the peak of taurine-modified uridine or unmodified guanosine.

FIG. 8 is a diagram showing the results of comparison of the amount of 2-methylthio-N6-isopentenyl adenosine (ms²i⁶A) and taurine-modified uridine in urine samples of patients with a mitochondrial disease (CEPO) and healthy individuals. As a control, the amount of guanosine, which is a representative of the unmodified base, was measured.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure as defined by the appended claims.

### DETAILED DESCRIPTION OF EXEMPLARY EMBODIMENTS

The exemplary embodiments of the present disclosure will be described in detail below, but the present disclosure is not limited to aspects or the exemplary embodiments that are described below.

Hereinafter, the present disclosure will be illustrated and described in detail with reference to the exemplary embodiments, along with the preferred methods and materials which can be used in practice of the present disclosure. However, the present disclosure is not limited thereto.

Unless otherwise specified in the sentences, any technical terms and scientific terms used in the present specification, have the same meaning as those generally understood by those of ordinary skill in the art to which the present disclosure belongs. Any materials and methods equivalent or similar to those described in the present specification can be used for practicing the present disclosure. All publications and patents cited herein in connection with the present disclosure described herein are incorporated herein by reference, for example, as indicating methodology, materials, etc. that can be used in the present disclosure.

In the present specification, “and/or” is used in a sense that any one or both are included.

Hereinafter, the present disclosure will be described in detail. The present disclosure relates to a method for determining the amount of a modified nucleoside (for example, the amount of taurine-modified uridine) in a sample by tandem mass spectrometry, a method for determining the amount of a modified nucleoside (for example, the amount of taurine-modified uridine) contained in mt-tRNA in a test animal, comprising determining the amount of a modified nucleoside (for example, taurine-modified uridine) in a sample derived from the test animal, and the like.

Hereinafter, the present disclosure will be described using taurine-modified uridine as an example of the modified nucleosides to be measured in the present disclosure, but the present disclosure is not limited thereto, and any modified nucleoside is also a measurement subject of the present disclosure.

For example, modified nucleosides that are measurement subjects of the present disclosure include, but are not limited to, 2-methylthio-N6-threonylcarbamoyl adenosine (ms²t⁶A), N6-methyladenosine (m⁵A) and the like, in addition to above-mentioned 5-taurinomethyl-2-thiouridine (τm⁵s²U), 5-taurinomethyluridine (τm⁵U)) and 2-methylthio-N6-isopentenyl adenosine (ms² i⁶A).

In the present disclosure, 5-taurinomethyl-2-thiouridine (τm⁵s²U) or 5-taurinomethyluridine (τm⁵U) may be collectively referred to as “taurine-modified uridine”.

5-taurinomethyl-2-thiouridine (τm⁵s²U)

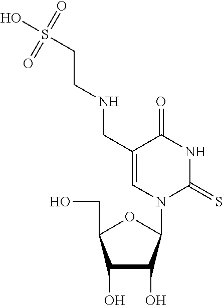

5-taurinomethyluridine (τm⁵U)

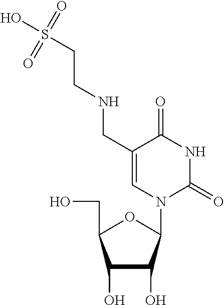

In the exemplary embodiments of the present disclosure, the phrase “the amount of taurine-modified uridine contained in mt-tRNA” refers to the amount of a uracil residue modified with taurine contained in mt-tRNA, and “mt-tRNA contains taurine-modified uridine” means that mt-tRNA has a uracil residue modified with taurine.

1. Method for Determining the Amount of Taurine-Modified Uridine in a Test Animal, Including Determining the Amount of Taurine-Modified Uridine in Urine of a Test Animal

The present disclosure provides a method for determining the amount of taurine-modified uridine in a sample by tandem mass spectrometry (in the present specification, also referred to as method 1 of the present disclosure).

The method 1 of the present disclosure contains a step of subjecting to liquid chromatography (LC) to obtain a fraction enriched in taurine-modified uridine.

In the present specification, chromatography refers to a technology that separates substances in the process in which the substance called mobile phase passes through the surface or inside of the substance called stationary phase (or carrier), and LC means chromatography in which the mobile phase is liquid. From the viewpoint of enhancing the resolution and detectability, it is preferable that LC in the present disclosure is high performance liquid chromatography (HPLC) (sometimes known as “high pressure liquid chromatography”).

In the present specification, the term “high performance liquid chromatography” refers to a method in which the liquid mobile phase is pressurized by a pump or the like to pass the stationary phase such as a high density packed column, and the analytes is separated and detected at high performance using a difference in interaction (adsorption, distribution, ion exchange, size exclusion, etc.) between the stationary phase and the mobile phase.

The type of chromatography which can be used for LC includes partition chromatography, normal phase liquid chromatography (NPLC), displacement chromatography, reverse phase liquid chromatography (RPLC), size exclusion chromatography, ion exchange chromatography, affinity chromatography and the like.

Although there is no limitation as long as a taurine-modified uridine-enriched fraction can be obtained, LC preferably used in the present disclosure includes RPLC.

In the present specification, the reverse phase liquid chromatography denotes chromatography using a polar mobile phase and a non-polar stationary phase.

In order to obtain a taurine-modified uridine-enriched fraction in the present disclosure, it is preferable to perform reverse phase HPLC, which is HPLC using RPLC as a separation mode, although it is not particularly limited as long as it can concentrate taurine-modified uridine.

Those skilled in the art can appropriately select a suitable analysis column used for LC, in accordance with the liquid chromatography method to be used and the target analyte and the like. The term “analysis column” used in the present specification means a chromatography column having sufficient properties to perform sufficient separation to allow determination of the presence or amount of the analyte in the sample.

In a preferred embodiment, the analysis column contains particles about 2 μm in diameter.

In the case of using reverse phase HPLC in the step (a), the filler used in the analysis column includes, for example, fillers in which a hydrocarbon chain is attached to a silica gel or polymer gel base. In the case of using reverse phase HPLC in the step (a), the preferable filler used in the analysis column includes fillers in which an octadecyl group is attached to a silica gel base (also referred to as ODS or C₁₈ filler).

Though there is no particular limitation as long as sufficient separation can be made to enable determination of the presence or amount of the analyte in a sample, for example, acetonitrile or methanol can be used as a solvent for elution.

Examples of the LC conditions include, but not limited to,

System: Shimazu LCMS 8050

Column: Inertsil ODS-3, 2 μm (GL Sciences)

Column length: 150 mm

Column inner diameter: 2.1 mm

Eluent: A) 30 mM ammonium acetate (pH 5.8):
B) 60% acetonitrile

Flow rate: 0.4 mL/min

Column temperature: 50° C.

Injection volume: 2 μL.

When detection of taurine-modified uridine is performed using a quadrupole mass spectrometer LCMS (LCMS-8050; manufactured by Shimadzu Corp.) under the above conditions, the peak of τm⁵U is detected at 2 minutes±10% and the peak of τm⁵s²U is detected at 3 minutes±10%.

The fractions enriched in taurine-modified uridine can be obtained also in different LC-MS-MS configurations by those skilled in the art with reference to the methods described in the examples and the like.

The method 1 of the present disclosure contains a step of subjecting the taurine-modified uridine-enriched fraction to an ionization source under conditions suitable for generating a taurine-modified uridine ion.

The method for ionizing the taurine-modified uridine-enriched fraction includes electro spray ionization (ESI), atmospheric pressure chemical ionization (APCI), atmospheric pressure photoionization (APPI), electron ionization (EI), fast atom bombardment (FAB)/liquid secondary ionization (LSIMS), matrix-assisted laser desorption ionization (MALDI), field ionization, field desorption, thermal spray/plasma spray ionization, particle beam ionization and the like. Those skilled in the art can select the ionization method based on the analyte to be measured, the type of the sample, the type of the detector, the choice of positive ion mode or negative ion mode, and the like. The method is not particularly limited as long as taurine-modified uridine can be detected, but in the method 1 of the present disclosure, ESI is preferably used.

When used in the present specification, the term “mass spectrometry” or “MS” refers to an analytical technique to identify compounds by their mass, in which ions are filtered, detected and/or measured based on the mass-to-charge ratio (m/z) of the ion.

Tandem MS (also called MS/MS) means a method of taking out only a specific ion (also called precursor ion) by a first analyzer, cleaving it by some means, and analyzing the generated fragment ion (also called product ion) with a second mass meter.

The cleaving means preferred in the present disclosure includes collisional excitation. One preferred embodiment of the present disclosure includes a step of determining the amount of the product ion fragmented by CID (Collision Induced Dissociation) of the precursor ion. In the present specification, collision induced dissociation refers to breaking some bonds of the precursor ion by causing collision between the selected precursor ion and a neutral molecule.

Tandem MS in method 1 of the present disclosure is usually performed using selective reaction monitoring (SRM) since even if a contaminant having the same retention time as the target analyte (taurine-modified uridine) and the same m/z value as the precursor ion is present in chromatography, the effect can be eliminated as long as the product ion having the same m/z value as the target analyte is not generated from the contaminant.

In the exemplary embodiment of the present disclosure, “selective reaction monitoring” or “SRM” means that in multi-stage mass spectrometry with two or more stages, the mass spectrometer is operated so as to continuously detect only the amount of the signal of a specific product ion generating from the analysis target compound, instead of acquiring a product ion spectrum. In SRM, the tandem mass spectrometry may be spatial (tandem mass spectrometry in space) or temporal (tandem mass spectrometry in time).

Ionization and MS can be performed using a mass spectrometer. Generally, the mass spectrometer is composed of a sample introduction part, an ionization part (ion source), a mass separation part (analyzer), a detection part (detector), a vacuum evacuation part (vacuum pump), an apparatus control part-data processing part (data system), and the like.

Examples of the analyzer used for tandem MS in the method 1 of the present disclosure include a triple quadrupole analyzer, an ion trap analyzer, a time-of-flight analyzer and the like. Preferred analyzers for use in tandem MS in the method 1 of the present disclosure include a triple quadrupole analyzer or a quadrupole time-of-flight (QTOF) analyzer. In the method 1 of the present disclosure, it is more preferable to perform tandem MS using a triple quadrupole analyzer, in view of the fact that commercial instrument platforms advantageous for SRM assays often use a triple quadrupole analyzer. The term “triple quadrupole” as used herein is meant to include use of not only quadrupoles but also multipoles and laminate electrodes in place of quadrupoles, as those skilled in the art generally understand.

In the exemplary embodiment of the present disclosure, the mass spectrometry may be performed by negative ion mode. Alternatively, the mass spectrometry may be performed by positive ion mode. When used in the present specification, the term “positive ion mode” refers to a mass spectrometry method in which positive ions are generated and detected, and the term “negative ion mode” refers to a mass spectrometry method in which negative ions are generated and detected.

In an exemplary embodiment of the method 1 of the present disclosure, taurine-modified uridine is analyzed by negative ion mode. In a more preferred embodiment of the method 1 of the present disclosure, taurine-modified uridine is ionized by ESI and analyzed by negative ion mode. Further, in an exemplary embodiment of the present disclosure, detection of ms2i6-modified adenosine is analyzed by positive ion mode.

The method 1 of the exemplary embodiment of the present disclosure contains a step (c) of determining the amount of taurine-modified uridine product ion (τm5s2U product ion and/or τm5U product ion) by tandem mass spectrometry. An exemplary embodiment of the method 1 of the present disclosure contains a step (c) of determining the amount of taurine-modified uridine product ion by tandem mass spectrometry and the tandem mass spectrometry in the step (c) includes subjecting precursor ions having the same m/z as the taurine-modified uridine precursor ion among ions ionized in the step (b) to a collision reaction under conditions in which the taurine-modified uridine precursor ion generates a taurine product ion and determining the amount of the product ion having the same m/z as the taurine product ion among the product ions generated by the collision reaction.

The amount of ions determined in the step (c) above is correlated with the amount of taurine-modified uridine in a sample. In one embodiment, the determination of the amount of taurine-modified uridine in a sample may be relative quantification. In one exemplary embodiment, the determination of the amount of taurine-modified uridine in a sample may be absolute quantification.

In one exemplary embodiment of the method 1 of the present disclosure, the step (c) of determining the amount of taurine-modified uridine ions by tandem mass spectrometry includes the following steps (c1), (c2) and (c3):

(c1) a step of selecting an ion having the same m/z as the taurine-modified uridine precursor ion (preferably, a negative ion having 396±0.5 m/z for detection of τm5s2U, and/or a negative ion having 380±0.5 m/z for detection of τm5U) among the ions ionized in the step (b),

(c2) a step of subjecting the ions selected in the step (c1) to a collision reaction under conditions in which the taurine-modified uridine precursor ion generates a taurine-modified uridine product ion (preferably, a taurine-modified uridine product negative ion having an m/z of 124±0.5),

(c3) a step of determining the amount of ions having the same m/z as the taurine-modified uridine product ion (preferably, a product ion having an m/z of 124±0.5) produced by the collision reaction in the step (c2),

wherein the amount of the ions determined in the step (c3) is correlated with the amount of taurine-modified uridine in the sample.

In any of the methods set forth in the exemplary embodiment of the present disclosure, one or more internal standards that can be separately detected may be provided in a sample, and this amount may also be determined in a sample. In one embodiment utilizing an individually detectable internal standard, all or part of both the target analyte and the internal standard present in a sample may be ionized to generate a plurality of ions that can be detected by a mass spectrometer, and one or more ions generated from them can be detected by mass spectrometry. In these embodiments, the presence or amount of ions generated from the target analyte can be correlated with the presence of the amount of the target analyte in a sample by comparison with the amount of the internal standard ion detected.

In one embodiment, the amount of the taurine-modified uridine ion can be correlated with the amount of taurine-modified uridine in a sample by comparison with the internal standard substance.

In another embodiment, the amount of the target analyte in a sample can be determined by comparison to one or more external reference standards. Examples of the external reference standard include samples spiked with taurine-modified uridine, and the like. The external reference standard is generally subjected to the same treatment and analysis as the other samples to be analyzed.

In one embodiment using SRM, the relative quantification of taurine modified uridine can be performed, for example, by an SMR method of comparing the SRM feature peak area of taurine-modified uridine in the different sample (for example, the feature peak area or integrated product ion intensity).

In another embodiment using SRM, the relative quantification of taurine modified uridine can be performed, for example, by comparing the SRM feature peak area of taurine modified uridine with the SRM feature peak area from different substances in the same sample (for example, unmodified uridine).

In one embodiment using SRM, the absolute quantification of taurine-modified uridine can be determined, for example, by an SRM method in which the SRM feature peak area of taurine modified uridine in one biological sample is compared to the SRM feature peak area of an externally added internal standard.

In one embodiment, the internal standard is a synthetic taurine-modified uridine labeled with one or more heavy isotopes. The appropriate isotope-labeled internal standard can be synthesized to generate a predictable and consistent SRM feature peak which, when analyzed by mass spectrometry, it can be used as a control peak as it is different and distinguishable from the natural taurine-modified uridine feature peak. Thus, when the sample is added with a known amount of internal standard and analyzed by mass spectrometry, the SRM feature peak area of the natural taurine modified uridine in the same sample can be compared to the SRM feature peak of the internal standard.

In the exemplary embodiment of the present disclosure, “sample” means any sample that can contain taurine-modified uridine. Although not particularly limited, a cell culture supernatant or a body fluid sample is preferably used as the sample. In one embodiment, the sample used in the present disclosure is of human origin.

In the exemplary embodiment of the present disclosure, “body fluid” means any fluid that can be isolated from the body of an individual. Examples of the body fluid include blood, plasma, serum, bile, saliva, urine, tears, sweat, cerebrospinal fluid (CSF) and the like. Preferably, the body fluid is urine or serum, most preferably urine.

In the exemplary embodiment of the present disclosure, “cell culture supernatant” means the supernatant of a culture obtained by culturing any cells (preferably animal cells, more preferably mammalian cells) in a culture medium for a certain period of time. Examples of cells that can be used for preparation of the culture supernatant include cultured cells, cells isolated from the body of an animal individual, or cells derived from cells isolated from the body of an animal individual such as pluripotent stem cells derived from cells isolated from the body of an animal individual or cells differentiated from the pluripotent stem cells, and the like. Though the culture conditions of the cells are not particularly limited as long as taurine-modified uridine can be detected, for example, 10⁵ to 10⁷ cells are seeded in a culture dish containing 2 mL of a culture medium for cell culture and cultured for 1 day or more to obtain the culture; the culture supernatant thereof and the like being mentioned.

The sample is preferably deproteinized from the viewpoint of preventing contaminating signals from being mixed in the measurement results by proteins in the sample. The method of “deproteinization” includes, generally, insolubilization by denaturation of proteins (addition of acids such as perchloric acid, trichloroacetic acid and metaphosphoric acid, addition of water-miscible organic solvents such as acetone, acetonitrile, methanol and ethanol, heating and cooling), and physical removal (ultrafiltration by membrane filter (centrifugal filtration device), dialysis by dialysis tube, ultracentrifugation) and the like. Further, deproteinization can also be carried out by using permeation limiting fillers such as internal reverse phase fillers, hybrid type fillers, hydrophilic polymer fillers and the like. There is no limitation as long as there is no problem in determining the amount of taurine-modified uridine, but examples of preferred deproteinization treatments include deproteinization using insolubilization by protein denaturation with an organic solvent miscible with water, and include, for example, deproteinization using methanol. The method of the deproteinization treatment is known and can be performed according to a standard method. Although not particularly limited, for example, in the deproteinization treatment, 0.2 to 20 times, preferably 1 to 5 times the amount of ethanol or methanol is added to a sample (preferably, a body fluid sample or a cell culture supernatant), and reacted for sufficient time for protein denaturation (for example, 15 minutes), then, centrifugation is carried out under conditions sufficient to precipitate the denatured protein (e.g., 12,000×g for 15 minutes), and the supernatant (organic solvent layer) is recovered, thus the deproteinized sample can be obtained. The deproteinized sample may be used as it is or after being dried by a centrifugal evaporator or the like, dissolved in an appropriate solvent such as distilled water, and used for LC.

Though the amount of the sample to be subjected to liquid chromatography is not particularly limited as long as it can detect taurine-modified uridine, for example, in the case of a deproteinized human urine sample, it is 1 to 100 μL. For example, by subjecting 1 to 10 μL of a human urine sample to liquid chromatography, it is possible to obtain a taurine-modified uridine-enriched fraction sufficient for detection of taurine-modified uridine.

As a more preferred embodiment of the method 1 of the present disclosure, exemplified is a method for determining the amount of taurine-modified uridine (τm⁵s²U and/or τm⁵U) in a sample (preferably a cell culture supernatant or body fluid sample, more preferably a body fluid sample, further preferably urine, preferably deproteinized, more preferably deproteinized with a water-miscible organic solvent, further preferably deproteinized with methanol) by tandem mass spectrometry, comprising

(a) a step of subjecting a sample suspected of containing taurine-modified uridine to LC (preferably HPLC, more preferably reverse phase HPLC) to obtain a fraction enriched in the taurine-modified uridine,

(b) a step of subjecting the taurine-modified uridine-enriched fraction to an ionization source (preferably an ESI ionization source) under conditions suitable to generate a taurine modified uridine precursor ion which can be detected by mass spectrometry,

(c1) a step of selecting an ion having the same m/z as the taurine-modified uridine precursor ion (a negative ion having 396±0.5 m/z for detection of τm⁵s²U, a negative ion having 380±0.5 m/z for detection of τm⁵U) among the ions ionized in the step (b),

(c2) a step of subjecting the ions selected in the step (c1) to a collision reaction under conditions in which the taurine-modified uridine precursor ion generates a taurine-modified uridine product negative ion (preferably, a taurine-modified uridine product negative ion having an m/z of 124±0.5),

(c3) a step of determining the amount of ions having the same m/z as the taurine-modified uridine product negative ion (that is, a product ion having an m/z of 124±0.5) produced by the collision reaction in the step (c2),

wherein the amount of the product ions determined in the step (c3) is correlated with the amount of taurine-modified uridine in the sample.

In one exemplary embodiment of the present disclosure, τm⁵s²U and τm⁵U in a sample can also be detected simultaneously.

2. Method for Determining the Amount of Taurine-Modified Uridine Contained in Mt-tRNA in a Test Animal

The exemplary embodiment of the present disclosure further provides a method for determining the amount of taurine-modified uridine contained in mt-tRNA in a test animal, comprising determining the amount of taurine modified uridine in a sample selected from the group consisting of a culture supernatant of cells derived from the test animal and a body fluid sample derived from the test animal, wherein the determined amount of taurine modified uridine in the sample is correlated with the amount of taurine modified uridine of mt-tRNA in the test animal (in the present specification, also referred to as method 2 of the present disclosure).

According to the method 2 of the present disclosure, by using a culture supernatant of cells derived from a test animal or a body fluid sample of a test animal, it may be possible to non-invasively determine the amount of taurine-modified uridine contained in mt-tRNA in cells in the test animal.

Examples of cells derived from a test animal include cells isolated from the test animal, pluripotent stem cells prepared using the cells isolated from the test animal, and cells differentiated from the cells, and the like.

In the method 2 of the present disclosure, the test animal to be measured includes, for example, mammals (e.g.: human, monkey, cow, pig, horse, dog, cat, sheep, goat, rabbit, hamster, guinea pig, mouse, rat, etc.), birds (e.g.: chicken, etc.) and the like, preferably mammals.

In one embodiment of the present disclosure, τm⁵s²U and τm⁵U in a sample can also be detected simultaneously.

For example, determination of the amount of taurine-modified uridine in a sample can be performed using mass spectrometry.

In one embodiment of the method 2 of the present disclosure, the determination of the amount of taurine-modified uridine in a sample comprises

(b′) a step of subjecting a taurine-modified uridine-enriched fraction to an ionization source under conditions suitable for generating a taurine-modified uridine ion which can be detected by mass spectrometry; and

(c′) a step of determining the amount of the taurine-modified uridine ion by mass spectrometry,

wherein the amount of the taurine-modified uridine ion determined in the step (c′) is correlated with the amount of taurine-modified uridine in the sample, that is, correlated with the amount of taurine-modified uridine of mt-tRNA in the test animal.

Though there is not particular limitation as long as the amount of taurine-modified uridine in a sample can be determined, the method of ionizing the taurine-modified uridine-enriched fraction includes ESI, APCI, APPI, EI, FAB/LSIMS, MALDI, a field ionization method, a field desorption method, a thermal spray/plasma spray ionization method, a particle beam-ionization method, and the like, and it is preferable to use ESI for the ionization.

The mass spectrometry may be performed in positive ion mode. Alternatively, the mass spectrometry may be performed in negative ion mode. In a preferred embodiment, taurine-modified uridine is ionized by ESI and analyzed in negative ion mode.

Ionization and MS can be performed using a mass spectrometer. When MS is used in the method 2 of the present disclosure, the analyzer used for MS is not particularly limited as long as the amount of taurine modified uridine in a sample can be determined, and examples thereof include quadrupole (Q) analyzers, triple quadrupole (QqQ) analyzers, Fourier transform ion cyclotron resonance (FTICR) analyzers, ion trap (IT) analyzer, time-of-flight (TOF) analyzers, hybrid tandem analyzers (Q-TOF, IT-TOF, Q-trap, Q-FTICR) and the like.

In one embodiment, the determination of the amount of taurine-modified uridine in a sample in the method 2 of the present disclosure can be performed using tandem mass spectrometry. When using tandem mass spectrometry in the method 2 of the present disclosure, the tandem mass spectrometry may be performed by any method known in the art including, for example, selective reaction monitoring, precursor ion scanning or product ion scanning. When determining the amount of taurine-modified uridine in a sample using tandem MS in the method 2 of the present disclosure, it is preferable to conduct selective reaction monitoring (SRM) since even if a contaminant having the same retention time as the target analyte (taurine-modified uridine) and the same m/z value as the precursor ion is present in chromatography, the influence of the contaminant can be eliminated as long as the product ion having the same m/z value as the target analyte is not generated from the contaminant. In SRM in the method 2 of the present disclosure, the tandem mass spectrometry may be spatial (tandem mass spectrometry in space) or temporal (tandem mass spectrometry in time).

Though there is no particular limitation as long as the amount of taurine-modified uridine can be determined, the analyzer used in mass spectrometry in the method 2 of the present disclosure is preferably a triple quadrupole analyzer or a QTOF analyzer, and when SRM assays are used, it is preferable to use a triple quadrupole analyzer as the analyzer in view of the fact that commercial equipment platforms advantageous for SRM assays often use a triple quadrupole analyzer.

In the determination of the amount of taurine-modified uridine in a sample by using the method 1 or 2 of the present disclosure, a sample prepared in the same manner as for the test sample except that it is substantially free of taurine-modified uridine may be used as a negative control sample. For example, a culture supernatant of cells in which the Mto-1 gene has been knocked out may be used as a negative control sample. In addition, a standard sample prepared in the same manner as for the test sample from a group of healthy animals not suffering from mitochondrial diseases may be used as a positive control sample. Furthermore, a sample to which taurine-modified uridine has been externally added can also be used as a positive control.

Based on the determined amount of taurine-modified uridine in a sample, the amount of taurine-modified uridine contained in mt-tRNA in a test animal can be determined.

The determination of the amount of taurine-modified uridine contained in mt-tRNA in a test animal can be performed, for example, using one or more internal standards provided in a sample.

In one embodiment, the amount of the taurine-modified uridine ion can be correlated with the amount of taurine-modified uridine contained in mt-tRNA in a test animal by comparison with the internal standard substance.

Alternatively, the determination of the amount of taurine-modified uridine contained in mt-tRNA in a test animal can be performed, for example, using one or more external reference standards provided in a sample.

The quantification using an internal standard or an external standard may be performed using a previously prepared calibration curve.

In the method 2 of the present disclosure, the determination of the amount of taurine-modified uridine contained in mt-tRNA in a test animal can be relative quantification.

As an exemplary embodiment of the method 2 of the present disclosure, exemplified is a method for determining the amount of taurine-modified uridine contained in mt-tRNA in a test animal, comprising

(a′) a step of obtaining a taurine-modified uridine-enriched fraction from one or more samples selected from the group consisting of a culture supernatant of cells derived from the test animal and a body fluid sample derived from the test animal (preferably a body fluid sample, more preferably urine, preferably deproteinized, more preferably deproteinized with a water-miscible organic solvent, further preferably deproteinized with methanol),

(b′) a step of subjecting the taurine-modified uridine-enriched fraction obtained in the step (a′) to an ionization source under conditions suitable for generating a taurine-modified uridine ion which can be detected by mass spectrometry; and

(c′) a step of determining the amount of the taurine-modified uridine ion by mass spectrometry,

wherein the amount of the ion determined in the step (c′) is correlated with the amount of taurine-modified uridine contained in mt-tRNA in the test animal.

The method for obtaining a taurine-modified uridine-enriched fraction in the above-described step (a′) is not particularly limited as long as sufficient separation can be performed to enable the determination of the amount of taurine-modified uridine in a sample, and the method can be practiced by any method known in the art. The taurine-modified uridine-enriched fraction can be obtained by performing any method such as, for example, liquid chromatography, filtration, centrifugation, thin layer chromatography, electrophoresis including capillary electrophoresis, affinity separation including immunoaffinity separation, and the like, or a combination thereof.

In one exemplary embodiment, the step (c′) of determining the amount of the taurine-modified uridine ions by tandem mass spectrometry comprises the following steps (c1′), (c2′) and (c3′):

(c1′) a step of selecting an ion having the same m/z as the taurine-modified uridine precursor ion (preferably, a negative ion having 396±0.5 m/z or a positive ion having 398±0.5 m/z for detection of τm⁵s²U, and/or a negative ion having 380±0.5 m/z, or a positive ion having 380±0.5 m/z or 382±0.5 m/z for detection of τm⁵U) among the ions ionized in the step (b′),

(c2′) a step of subjecting the ions selected in the step (c1′) to a collision reaction under conditions in which the taurine-modified uridine precursor ion generates a taurine-modified uridine product ion (preferably, a taurine-modified uridine product negative ion having an m/z of 124±0.5 when the ion selected in the step (c1′) is a negative ion having 396±0.5 m/z and/or a negative ion having 380±0.5 m/z; a taurine-modified uridine product positive ion having an m/z of 126±0.5 and/or 266±0.5 when the ion selected in the step (c1′) is a positive ion having 398±0.5 m/z; a taurine-modified uridine product positive ion having 126±0.5 when the ion selected in the step (c1′) is a positive ion having 380±0.5 m/z; a taurine-modified uridine product positive ion having an m/z of 250±0.5 when the ion selected in the step (c1′) is a positive ion having 382±0.5 m/z),

(c3′) a step of determining the amount of ions having the same m/z as the taurine-modified uridine product ion (preferably, an ion having an m/z of 124±0.5 when the ion selected in the step (c1′) is a negative ion having 396±0.5 m/z and/or a negative ion having 380±0.5 m/z; a positive ion having an m/z of 126±0.5 and/or 266±0.5 when the ion selected in the step (c1′) is a positive ion having 398±0.5 m/z; a positive ion having an m/z of 126±0.5 when the ion selected in the step (c1′) is a positive ion having 380±0.5 m/z; a positive ion having an m/z of 250±0.5 when the ion selected in the step (c1′) is a positive ion having 382±0.5 m/z) produced by the collision reaction in the step (c2′),

wherein the amount of the ions determined in the step (c3) is correlated with the amount of taurine-modified uridine in the sample.

As one exemplary embodiment of the method 2 of the present disclosure, exemplified is a method for determining the amount of taurine-modified uridine contained in mt-tRNA in a test animal, comprising

(a″) a step of subjecting one or more samples selected from the group consisting of a culture supernatant of cells derived from the test animal and a body fluid sample derived from the test animal (preferably a body fluid sample, more preferably urine, preferably deproteinized, more preferably deproteinized with a water-miscible organic solvent, further preferably deproteinized with methanol) to LC (preferably HPLC) to obtain a taurine-modified uridine-enriched fraction,

(b′) a step of subjecting the taurine-modified uridine-enriched fraction obtained in the step (a″) to an ionization source under conditions suitable for generating a taurine-modified uridine ion which can be detected by mass spectrometry; and

(c′) a step of determining the amount of the taurine-modified uridine ion by mass spectrometry,

wherein the amount of the ion determined in the step (c′) is correlated with the amount of taurine-modified uridine contained in mt-tRNA in the test animal.

As one exemplary embodiment of the method 2 of the present disclosure, exemplified is a method for determining the amount of taurine-modified uridine (τm⁵s²U and/or τm⁵U) contained in mt-tRNA in a test animal, comprising

(a′) a step of obtaining a taurine-modified uridine-enriched fraction from one or more samples selected from the group consisting of a culture supernatant of cells derived from the test animal and a body fluid sample derived from the test animal (preferably a body fluid sample, more preferably urine, preferably deproteinized, more preferably deproteinized with a water-miscible organic solvent, further preferably deproteinized with methanol)(preferably, subjecting the sample to LC to obtain a taurine-modified uridine-enriched fraction),

(b′) a step of subjecting the taurine-modified uridine-enriched fraction obtained in the step (a′) to an ionization source (preferably an ESI ionization source) under conditions suitable for generating a taurine-modified uridine precursor ion which can be detected by mass spectrometry;

(c1′) a step of selecting an ion having the same m/z as the taurine-modified uridine precursor ion (preferably, a negative ion having 396±0.5 m/z or a positive ion having 398±0.5 m/z for detection of τm⁵s²U, and/or a negative ion having 380±0.5 m/z, or a positive ion having 380±0.5 m/z or 382±0.5 m/z for detection of τm⁵U) among the ions ionized in the step (b′),

(c2′) a step of subjecting the ions selected in the step (c1′) to a collision reaction under conditions in which the taurine-modified uridine precursor ion generates a taurine-modified uridine product ion (preferably, a taurine-modified uridine product negative ion having an m/z of 124±0.5 when the ion selected in the step (c1′) is a negative ion having 396±0.5 m/z and/or a negative ion having 380±0.5 m/z; a taurine-modified uridine product positive ion having an m/z of 126±0.5 and/or 266±0.5 when the ion selected in the step (c1′) is a positive ion having 398±0.5 m/z; a taurine-modified uridine product positive ion having 126±0.5 when the ion selected in the step (c1′) is a positive ion having 380±0.5 m/z; a taurine-modified uridine product positive ion having an m/z of 250±0.5 when the ion selected in the step (c1′) is a positive ion having 382±0.5 m/z),

(c3′) a step of determining the amount of ions having the same m/z as the taurine-modified uridine product ion (preferably, an ion having an m/z of 124±0.5 when the ion selected in the step (c1′) is a negative ion having 396±0.5 m/z and/or a negative ion having 380±0.5 m/z; a positive ion having an m/z of 126±0.5 and/or 266±0.5 when the ion selected in the step (c1′) is a positive ion having 398±0.5 m/z; a positive ion having 126±0.5 when the ion selected in the step (c1′) is a positive ion having 380±0.5 m/z; a positive ion having an m/z of 250±0.5 when the ion selected in the step (c1′) is a positive ion having 382±0.5 m/z) produced by the collision reaction in the step (c2′),

wherein the amount of the ions determined in the step (c3′) is correlated with the amount of taurine-modified uridine in the sample.

When obtaining a taurine-modified uridine-enriched fraction by LC in the step (a′) of the method 2 of the present disclosure, the amount of the sample subjected to liquid chromatography is not particularly limited as long as taurine-modified uridine can be detected, and for example, 1 to 100 μL of a deproteinized human urine sample is mentioned. For example, by subjecting 1 to 10 μL of a human urine sample to liquid chromatography, it is possible to obtain a taurine-modified uridine-enriched fraction which is sufficient for detection of taurine-modified uridine.

In an exemplary embodiment of the method 2 of the present disclosure, the determination of the amount of taurine-modified uridine in a sample is performed using the method 1 of the present disclosure.

3. Method for Examining Possibility of Onset of Mitochondrial Disease in Test Animal

The present disclosure further provides an exemplary method for examining the possibility of onset of mitochondrial diseases based on the negative correlation between the amount of taurine-modified uridine contained in mt-tRNA in a test animal determined by the method 2 of the present disclosure and the possibility of onset of mitochondrial diseases (in the present specification, also referred to as method 3 of the present disclosure).

In humans with mitochondrial diseases, it is known that the amount of taurine-modified uridine in mt-tRNA is reduced as compared to humans without mitochondrial diseases. That is, the possibility of onset of mitochondrial diseases (preferably mitochondrial diseases caused by a reduction in the amount of taurine modified uridine) can be examined based on the negative correlation between taurine-modified uridine and the possibility of onset of mitochondrial diseases.

For example, when the amount of taurine-modified uridine contained in mt-tRNA of a test animal (for example, a human subject to be tested) is relatively low as compared to that of a negative control group not having a mitochondrial disease (for example, a human not having a mitochondrial disease), it can be determined that the test animal has high possibility of onset of mitochondrial diseases. Therefore, the possibility of onset of mitochondrial diseases in a test animal can be examined, by comparing the amount of taurine-modified uridine contained in mt-tRNA in the test animal determined by the method 2 of the present disclosure with such judgment criteria.

In addition, the cutoff value of the amount of taurine-modified uridine contained in mt-tRNA may be set in advance, and the amount of taurine-modified uridine contained in mt-tRNA determined in method 2 of the present disclosure and this cutoff value may be compared. For example, if the amount of taurine-modified uridine contained in mt-tRNA of a test animal determined in the method 2 of the present disclosure is lower than the cutoff value, it can be determined that the test animal has high possibility of onset of mitochondrial diseases.

The “cut-off value” is a value that can satisfy both high diagnostic sensitivity and high diagnostic specificity when determining the onset of a disease based on that value. For example, the amount of taurine-modified uridine in a sample that exhibits a high positive rate in humans with mitochondrial diseases and a high negative rate in humans showing no onset of mitochondrial disease can be set as the cutoff value.

Methods for calculating the cutoff value are well known in the art. For example, the amounts of taurine-modified uridine contained in mt-tRNA of humans with mitochondrial diseases and humans showing no onset of mitochondrial diseases are measured, and the diagnostic sensitivity and diagnostic specificity at the measured values are determined, and based on these values, a ROC (Receiver Operating Characteristic) curve is created using commercially available analysis software. Then, a value when the diagnostic sensitivity and the diagnostic specificity are as close to 100% as possible can be obtained and used as the cutoff value.

Further, for example, the diagnostic efficiency at the detected value (the proportion of the total number of a case in which a person with mitochondrial disease was correctly judged as “mitochondrial disease” and a case in which a person showing no onset of mitochondrial disease was correctly judged as “no onset of mitochondrial disease” with respect to the total number of cases) can be obtained, and the value at which the highest diagnostic efficiency is calculated can be taken as the cutoff value.

In addition to the amount of one taurine-modified uridine in a sample, another amount of taurine-modified uridine or another indicator (for example, thiomethylating modification of mitochondrial tRNA (ms²i⁶A) (Wei et al. Cell Metab. 21, 428, 2015)) can be combined, and correlated with the onset risk of a mitochondrial disease, thus, it can be expected to determine the risk of onset of a mitochondrial disease with higher accuracy.

Using the method 3 of the present disclosure, a method of identifying a test animal predisposed to mitochondrial diseases or a method of collecting data for the assessment of diagnosis or predisposition of mitochondrial diseases may be provided.

Unless otherwise stated, the definition of each term is the same as that described in above 1 or 2.

Although the invention has been described by way of example for the detection of taurine-modified uridine as described above, it is clear to those skilled in the art that the invention can be used for the detection of other modified nucleosides as well.

The exemplary embodiments of the present disclosure will be more specifically described by the following examples, which are merely illustrative of the present disclosure and do not limit the scope of the present disclosure.

### EXAMPLES

### Example 1: Detection of Taurine Modification by Selective Reaction Monitoring Method

In order to validate the detection method of τm⁵U and τm⁵s²U by mass spectrometry applying selective reaction monitoring, total RNA was extracted from wild type embryonic stem (ES) cells (WT cells) and ES cells deficient in Mtol, a taurine modification enzyme, (Mto1-KO cells), and injected into a quadrupole mass spectrometer LCMS manufactured by Shimadzu Corp. (LCMS-8050), and taurine modification was measured by a selective reaction monitoring method. Mto1-KO cells were prepared by deleting exons 3-4 of Mto1 gene by a homologous recombination method.

10⁶ cells were suspended in 1 mL of Trizol solution (Invitrogen), 0.2 mL of chloroform was added, and the mixture was centrifuged at 12,000×g for 15 minutes to separate total RNA into an aqueous phase. Thereafter, 0.5 mL of isopropanol was added to deposit total RNA, and the mixture was centrifuged at 12,000×g for 15 minutes to precipitate total RNA. The precipitate was washed once with 70% ethanol, and the total RNA was completely dried, and then the total RNA was redissolved with an appropriate amount of pure water.

As a separation column, reverse phase column Inertsil ODS-3 (150 mm×2.1 mm I.D., 2 μm) was used. Using mobile phase A: 30 mM ammonium acetate (pH 5.8) and mobile phase B: 60% acetonitrile, gradient analysis (mobile phase B: 0 min 1%→10 min 35%→15 min 100%→20 min 100%→25 min 1%) was carried out. The flow rate was set to 0.4 mL/min, and the column oven temperature was set to 50° C. The sample injection amount was 2 μL. The measurement was performed in negative ion mode.

Mass spectrometry was performed under the following conditions:

Ionization method (ESI)

Nebulizer gas flow rate: 3 L/min

Interface temperature: 300 degrees

Heat block temperature: 400 degrees

Dry in gas flow rate: 10 L/hr

Collision energy (CE): 25

For analysis of τm⁵U, a precursor ion with an m/z value of 380 was selected, and a cleavage reaction was caused by collision with an inert gas to further fragment it, and a peak of m/z 124 indicating a product ion was observed. In addition, in analysis of τm⁵s²U, a precursor ion having an m/z value of 396 was selected, and a cleavage reaction was caused by collision with an inert gas to further fragment it, and a peak of m/z 124 indicating a product ion was observed.

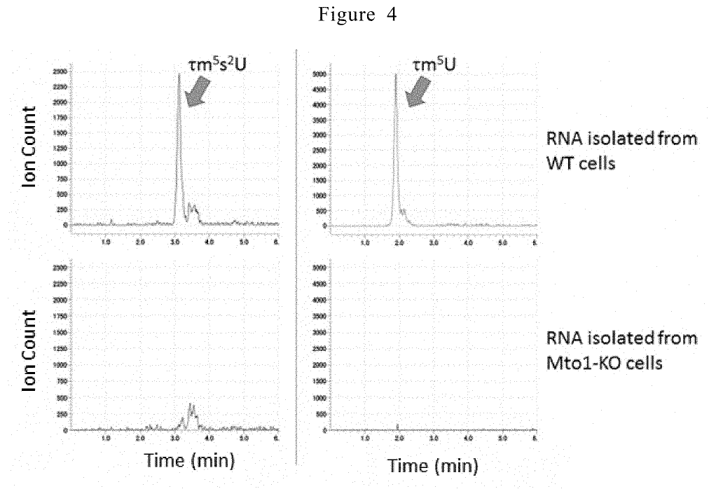

As a result, in RNA derived from normal cells, peaks corresponding to τm⁵s²U and τm⁵U were detected 3 minutes and 2 minutes after the start of analysis. Since these peaks disappeared in RNA derived from cells deficient in the taurine modification enzyme Mto1, these peaks were demonstrated to correspond to τm⁵s²U and τm⁵U (see FIG. 4).

### Example 2: Detection of Taurine Modification from Cell Culture Supernatant

Next, detection of taurine modification was performed using a cell culture supernatant. HeLa cells (1.5×10⁵ cells), which are human-derived cultured cells, were seeded on a 3.5 cm-diameter culture dish and cultured in 2 mL of DMEM (Dulbecco's Modified Eagle Medium) medium overnight. To 100 μL of the cell culture supernatant, 500 μL of methanol was added to extract taurine-modified nucleosides. The sample was centrifuged at 15000 rpm for 10 minutes in a centrifugal evaporator and dried, and then 100 μL of distilled water was added to re-dissolve. A 2 μL sample was injected into a quadrupole mass spectrometer LCMS (LCMS-8050) manufactured by Shimadzu Corp., and taurine modification was measured by the selective reaction monitoring method under the same conditions as in Example 1.

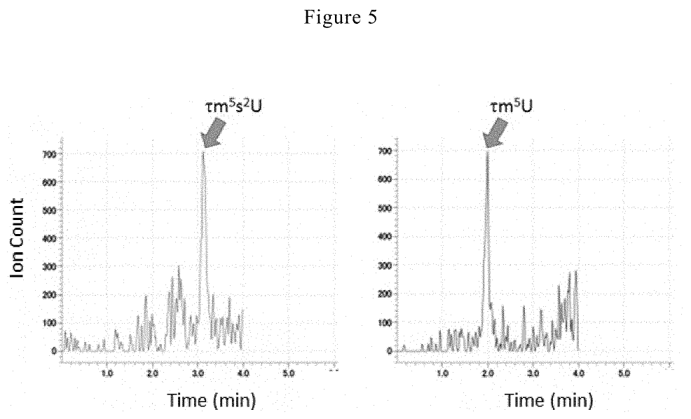

The exemplary results are shown in FIG. 5. By taurine modification analysis using a mass spectrometer, it was found that τm⁵U and τm⁵s²U can be detected from the culture supernatant.

### Example 3: Detection of Taurine Modification from Urine Sample

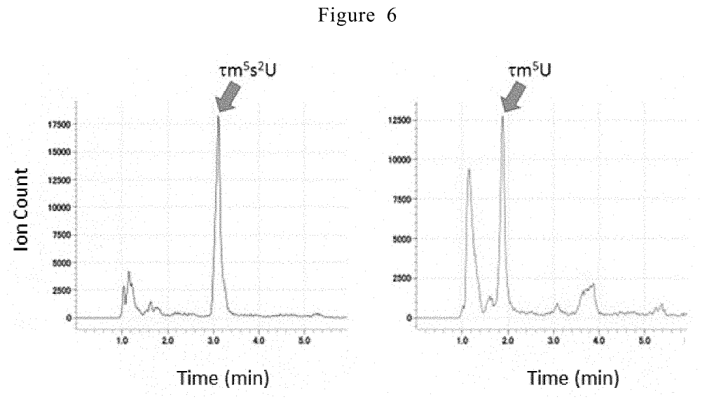

Based on the results of Example 2, detection of taurine modification using a biological sample was attempted. One hundred (100) μL of a human urine sample was collected, and 500 μL of methanol was added to carry out deproteinization. Next, it was centrifuged at 15000 rpm for 10 minutes, and the supernatant was dried by a centrifugal concentrator. Finally, the precipitate was dissolved in 100 μL of distilled water, and 2 μL thereof was analyzed with a mass spectrometer (LCMS-8050, manufactured by Shimadzu Corporation). The exemplary results are shown in FIG. 6

### Example 4: Detection of Taurine Modification in Patient with Mitochondrial Disease

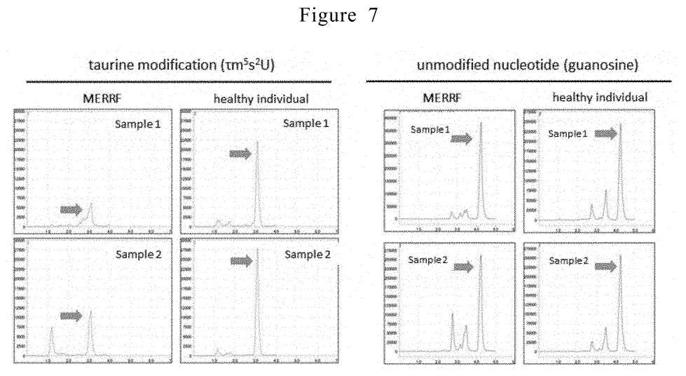

Urine was collected from two patients myoclonus epilepsy associated with ragged-red fiber (MERRF), which is one of mitochondrial diseases, and taurine modification (τm⁵s²U) in urine was analyzed by mass spectrometry. Urine was collected from 2 healthy individuals as control, and taurine modification (τm⁵s²U) was similarly analyzed by mass spectrometry. The exemplary results are shown in FIG. 7.

The τm⁵s²U dropped significantly in MERRF patients compared to healthy individuals. On the other hand, the unmodified nucleoside guanosine (G) was not different between MERRF patients and healthy individuals.

### Example 5: Detection of Taurine Modification from Urine Samples

The sample was prepared in the same manner as in Example 2, and mass analysis was performed in positive ion mode. For the detection of τm⁵s²U, a precursor positive ion having 398 m/z was selected, and a product positive ion of 126 m/z was detected. In the detection of τm⁵s²U, a precursor positive ion having 380 m/z was selected, and a product positive ion of 126 m/z was detected.

Taurine modification was also detected in the positive ion mode, but its detection sensitivity was lower when it was detected in the positive ion mode than in the negative ion mode.

### Example 6: Detection of Other Modified Nucleoside from Urine Sample of Patient with Other Mitochondrial Disease

The amounts of the modified nucleosides ms²i⁶A, and τm⁵s²U and τm⁵U were measured in patients with chronic external progressive ophthalmoplegia (CEPO), which is another mitochondrial disease.

CEPO is a type of mitochondrial disease shoes main symptoms are extraocular muscle paralysis, ptosis and limb weakness. 70% of CEPO show deletion or duplication of mitochondrial DNA. The region of mitochondrial DNA to be deleted varies from patient to patient. Southern blot and Long PCR, which detect deletion and duplication of mtDNA, are used in combination for gene diagnosis of CEPO. On the other hand, in the case of CEPO, since deletion of mitochondrial DNA may be found only in the affected eye muscle, there is a problem that not only blood but also eye muscle to be subjected to biopsy is required, which places a heavy burden on the patient.

Urine was collected from CEPO, and ms²i⁶A, and τm⁵s²U and τm⁵U in urine were analyzed by mass spectrometry. Urine was collected from healthy individuals as control, and similarly modified nucleosides were analyzed by mass spectrometry. Specifically, it was carried out as follows.

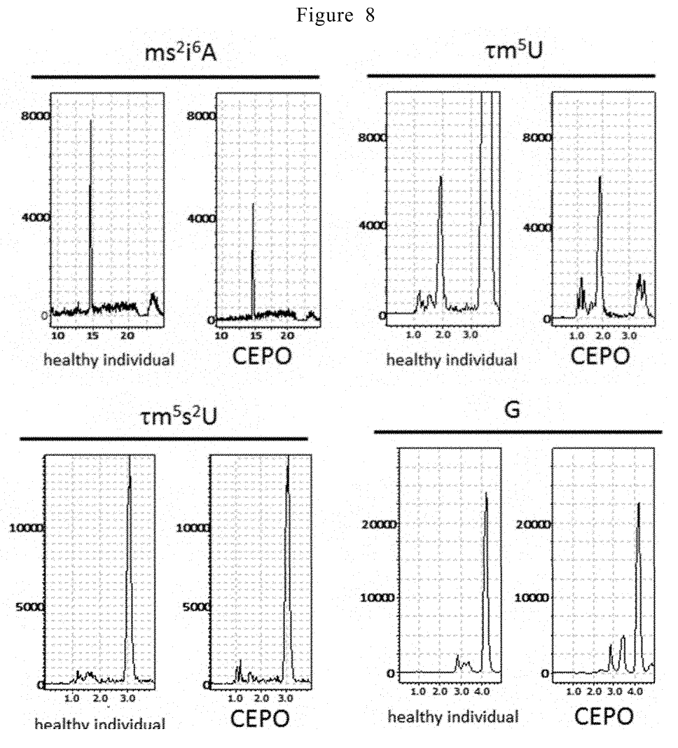

Deproteinization was performed by adding 500 μL of methanol to 100 μL of a human urine sample, followed by centrifugation at 15,000 rpm for 10 minutes, and collecting the supernatant. The collected supernatant was dried by a centrifugal evaporator, finally the precipitate was dissolved in 100 μL of distilled water, and 2 μL thereof was analyzed by a mass spectrometer (LCMS-8050, manufactured by Shimadzu Corporation). ms²i⁶A was detected by a selective reaction monitoring method. Detection parameters are as follows. ms²i⁶A: precursor positive ion m/z 382, product ion m/z 182; G: precursor positive ion m/z 284, product ion m/z 152. The detection parameters of τm⁵s²U and τm⁵U are the same as in Example 1. The results are shown in FIG. 8.

There was a marked decrease in ms²i⁶A in CEPO patients compared to healthy individuals. On the other hand, the τm⁵s²U and τm⁵U derived from the same mitochondrial tRNA and the unmodified nucleoside guanosine (G) did not differ between CEPO patients and healthy individuals.

According to the method for detecting modified nucleosides (for example, ms²i⁶A and taurine modified uridine) provided in the present disclosure, it may be possible to specifically detect modified nucleosides (for example, ms²i⁶A and taurine-modified uridine) from samples containing various substances such as body fluid samples such as urine and cell culture supernatants. Furthermore, according to the method for detecting modified nucleosides in a test animal provided in the present disclosure, it may be possible to non-invasively detect modified nucleosides from biological samples such as urine without painful muscle biopsy. Moreover, according to the detection method, it is possible to detect modified nucleosides from small amounts of samples, and all pretreatments are completed with only a few steps of centrifugation and concentration, hence, the method is very simple, the cost thereof is low, and anyone can get stable results. Furthermore, the functional analysis of mitochondria and diagnosis of mitochondrial diseases may be possible by using the method for detecting modified nucleosides provided in the present disclosure.

### EXEMPLARY REFERENCE(S)

The following reference is hereby incorporated by reference in its entirety, as follows:
Wei, F. Y. et al. Cdk5rap1-mediated 2-methylthio modification of mitochondrial tRNAs governs protein translation and contribute to myopathy in mice and humans. Cell Metab. 21: 428-442 (2015).

1. A method for determining the amount of modified nucleosides contained in mitochondrial tRNA (mt-tRNA) in a test animal, comprising:
(i) determining the amount of modified nucleosides in a sample selected from the group consisting of a body fluid sample derived from the test animal and a culture supernatant of cells derived from the test animal using tandem mass spectrometry; and
(ii) correlating the amount of the modified nucleosides in the sample determined by the step (i) with the amount of the modified nucleosides in mt-tRNA in the test animal.

2. The method according to claim 1, wherein the tandem mass spectrometry is LC-ESI-MS/MS, which includes liquid chromatography (LC) as a pretreatment and in which an ionization source is an electrospray ionization source (ESI), and an mass spectrometry mode to be used is a selective reaction monitoring.

3. The method according to claim 1, wherein the sample is urine derived from the test animal.

4. The method according to claim 3, wherein the sample is a urine sample deproteinized by methanol.

5. The method according to claim 3, wherein the sample is a urine sample from a human suspected of having a mitochondrial disease.

6. The method according to claim 5, further comprising:
(iii) correlating the amount of the modified nucleosides in mt-tRNA in the test animal correlated according to step (ii) with the degree of the mitochondrial disease in the test animal.

7. The method according to claim 1, wherein the modified nucleoside is taurine-modified uridine.

8. The method according to claim 7, wherein the taurine-modified uridine is 5-taurinomethyl 2-thiouridine (τm⁵s²U).

9. The method according to claim 8, wherein step (i) comprises substeps of:
(a) subjecting a sample suspected to contain τm⁵s²U to liquid chromatography to obtain a fraction enriched in τm⁵s²U,
(b) subjecting the fraction enriched in τm⁵s²U to an ionization source under conditions suitable to generate a τm⁵s²U precursor ion, and
(c) determining the amount of τm⁵s²U generated ions by tandem mass spectrometry (MS/MS),
wherein the tandem mass spectrometry (MS/MS) in the substep (c) includes subjecting the precursor negative ions having a mass-to-charge ratio (m/z) of 396±0.5 to a collision reaction under the condition,
wherein the τm⁵s²U precursor negative ions generate τm⁵s²U generated negative ions having an m/z of 124±0.5, to determine the amount of the generated ions having an m/z of 124±0.5 generated by the collision reaction,
and wherein the amount of ions determined by substep (c) is correlated with the amount of τm⁵s²U in the sample.

10. The method according to claim 9, wherein the sample is a urine sample obtained by deproteinizing urine derived from the test animal with methanol.

11. The method according to claim 7, wherein the taurine-modified uridine is 5-taurinomethyluridine (τm⁵U).

12. The method according to claim 11, wherein step (i) comprises substeps of:
(a) subjecting a sample suspected to contain τm⁵U to liquid chromatography to obtain a fraction enriched in τm⁵U,
(b) subjecting the fraction enriched in τm⁵U to an ionization source under conditions suitable to generate a τm⁵U precursor ion, and
(c) determining the amount of τm⁵U generated ions by tandem mass spectrometry (MS/MS),
wherein the tandem mass spectrometry (MS/MS) in substep (c) includes subjecting the precursor negative ions having a mass-to-charge ratio (m/z) of 380±0.5 to a collision reaction under the condition,
wherein the τm⁵U precursor negative ions generate τm⁵U generated negative ions having an m/z of 124±0.5, to determine the amount of the generated ions having an m/z of 124±0.5 generated by the collision reaction,
and wherein the amount of ions determined by substep (c) is correlated with the amount of τm⁵U in the sample.

13. The method according to claim 12, wherein the sample is a urine sample obtained by deproteinizing urine derived from the test animal with methanol.

14. The method according to claim 1, wherein the modified nucleoside is 2-methylthio-N6-isopentenyl adenosine (ms²i⁶A).

15. The method according to claim 14, wherein step (i) comprises substeps of:
(a) subjecting a sample suspected to contain ms²i⁶A to liquid chromatography to obtain a fraction enriched in ms²i⁶A,
(b) subjecting the fraction enriched in ms²i⁶A to an ionization source under conditions suitable to generate a ms²i⁶A precursor ion, and
(c) determining the amount of ms²i⁶A generated ions by tandem mass spectrometry (MS/MS),
wherein the tandem mass spectrometry (MS/MS) in substep (c) includes subjecting precursor positive ions having a mass-to-charge ratio (m/z) of 382±0.5 to a collision reaction under the condition,
wherein the ms²i⁶A precursor positive ions generate ms²i⁶A generated positive ions having an m/z of 182±0.5, to determine the amount of the generated ions having an m/z of 182±0.5 generated by the collision reaction,
and wherein the amount of ions determined by substep (c) is correlated with the amount of ms²i⁶A in the sample.

16. The method according to claim 15, wherein the sample is a urine sample obtained by deproteinizing urine derived from the test animal with methanol.

17. A method for determining an amount of τm⁵s²U in a sample by tandem mass spectrometry, comprising
(a) subjecting the sample suspected to contain τm⁵s²U to liquid chromatography to obtain a fraction enriched in τm⁵s²U,
(b) subjecting the fraction enriched in τm⁵s²U to an ionization source under conditions suitable to generate a τm⁵s²U precursor ion, and
(c) determining the amount of τm⁵s²U generated ions by the tandem mass spectrometry,
wherein the tandem mass spectrometry in step (c) includes subjecting precursor negative ions having a mass-to-charge ratio (m/z) of 396±0.5 to a collision reaction under the condition,
wherein the τm⁵s²U precursor negative ions generate τm⁵s²U generated negative ions having an m/z of 124±0.5, to determine the amount of the generated ions having an m/z of 124±0.5 generated by the collision reaction,
and wherein the amount of ions determined by step (c) is correlated with the amount of τm⁵s²U in the sample.

18. A method for determining an amount of τm⁵U in a sample by tandem mass spectrometry, comprising
(a) subjecting the sample suspected to contain τm⁵U to LC to obtain a fraction enriched in τm⁵U,
(b) subjecting the fraction enriched in τm⁵U to an ionization source under conditions suitable to generate a τm⁵U precursor ion, and
(c) determining the amount of τm⁵U generated ions by tandem mass spectrometry,
wherein the tandem mass spectrometry in step (c) includes subjecting precursor negative ions having a mass-to-charge ratio (m/z) of 380±0.5 to a collision reaction under the condition,
wherein the τm⁵U precursor negative ions generate τm⁵U generated negative ions having an m/z of 124±0.5, to determine the amount of the generated ions having an m/z of 124±0.5 generated by the collision reaction,
and wherein the amount of ions determined by step (c) is correlated with the amount of τm⁵U in the sample.

19. A method for determining the amount of ms²i⁶A in a sample by tandem mass spectrometry, comprising
(a) subjecting the sample suspected to contain ms²i⁶A to LC to obtain a fraction enriched in ms²i⁶A,
(b) subjecting the fraction enriched in ms²i⁶A to an ionization source under conditions suitable to generate a ms²i⁶A precursor ion, and
(c) determining the amount of ms²i⁶A generated ions by tandem mass spectrometry,
wherein the tandem mass spectrometry in step (c) includes subjecting precursor positive ions having a mass-to-charge ratio (m/z) of 382±0.5 to a collision reaction under the condition wherein the ms²i⁶A precursor positive ions generate ms²i⁶A generated positive ions having an m/z of 182±0.5, to determine the amount of the generated ions having an m/z of 182±0.5 generated by the collision reaction,
and wherein the amount of ions determined by step (c) is correlated with the amount of ms²i⁶A in the sample.

20-23. (canceled)

24. The method according to claim 19, wherein the sample is a body fluid sample derived from the test animal.
